(19) United States
(12) Patent Application Publication
(10) Pub. No.: US 2003/0208258 A1
(43) Pub. Date: Nov. 6, 2003

(54) Vascular grafts surrounding an expanded stent for transplantation

(76) Inventors: Tom Reilly, Wilmington, DE (US); Vincent A. Piccone JR., Staten Island, NY (US)

Correspondence Address: Louis A. Piccone, 245 Norht Gannon Ave., Staten Island, NY, 10314, US

(21) Appl. No.: 10/137,721

(22) Filed: May 2, 2002

(51) Int. Cl. A61F 2/06

(52) U.S. Cl. 623/1.13

(57) ABSTRACT

The present invention is directed to a vascular graft surrounding an expanded stent for transplantation into a patient.

### FIELD OF THE INVENTION

The present invention relates to the field of vascular tissue for re-transplantation into a patient. In particular, the present invention is directed to an in vitro tubular vascular graft surrounding an expanded stent for re-transplantation into a patient.

### BACKGROUND OF THE INVENTION

One of the most widely used cardiovascular surgical procedures is that of venous bypass grafting for the surgical treatment of occluded arteries including narrowed coronary arteries. Currently, autologous vein, saphaneous veins and mammary arteries are the prime choice for use as grafts for by-ass of periheral arteries and for aortocoronary re-vascularization. However, limited availability of first choice autologous bypass material and an increasing frequency of reoperations often mandate the use of less satisfactory autologous graft material. Such bypass grafts are prone to accelerated atherosclerosis and occlusion rates that can reach greater than 15% within the first year of surgery (Motwani, J. G., Topol, E. J. Circulation, 1998. 97:916-9310) and greater than 50% at 10 years after implantation (Capmeau, L., Enjalbert, M., Lesperance, J. et. al. Circulation. 1983. 68(suppl II): 1-7). This progressively severe narrowing is related to a cellular proliferation of smooth muscle, first in the medial layer of the blood vessel and finally also in the intima. The intima becomes thickened thus narrowing the blood vessel. In addition, up to 30% of all saphenous veins used for grafting are of inadequate quality (Huynh, T., Abraham, G., Murray, J. et. al. Nat. Biotechnol. 1999. 17:1083-1086).

In view of these problems, enormous efforts have been made to produce alternative small-caliber grafts with acceptable patency rates. For example, several tissue-engineered grafts using biotechnology have been discussed. However numerous technical, legal and ethical issues will delay their clinical introduction. In summary, because of its immense clinical importance, the search for an ideal small-caliber graft has been compared with the search for the holy grail (Conte, M.s., FASEB J. 1998: 12:43-45). Therefore, an important and immediate need exists for procedures by which grafts can be prepared to limit their occlusion rates following surgical transplantation.

The use of cryopreserved venous allografts, while established in bypass surgery, shows poor longevity for two primary reasons. The first problem is immunologically conditioned degeneration of the graft or immune rejection (Carpenter, J. P., Tomaszewski, J. E. J. Vasc. Surg. 1998. 27:492-499). Secondly, premature thrombotic occlusions are often observed. These 2 problems have been tracked back to either the presence or absence of the donor endothelium on the graft. A major role has recently been assigned to the endothelium as the culprit for acute and chronic organ rejection (Rose, M. L. Vasc. Med. 1997. 2:105-114). The release of non-HLA antigens by endothelium damaged during the cryopreservation process can lead to a chronic immune reaction and graft rejection. Consequently, some clinical investigators have advocated the complete removal of the donor endothelium from a graft before surgical placement into a recipient. However, such de-endothelialization can promote thrombotic events following surgical grafting. Thrombosis occurs as a result of the interactions of 3 primary factors: (1) aggregation of blood platelets, (2) activation of the co-agulation cascade; and, (3) smooth muscle cell migration and proliferation that also facilitates the development of accelerated atherosclerosis. Normally, vascular endothelium counterbalances the thrombotic tendancies through numerous anti-aggregatory and anti-coagulatory mechanisms. However, grafts which have been de-endothelialized lose this natural anti-thrombotic protection, and are prone to develop thrombosis and atherosclerosis.

Current procedures for preparing venous allografts for surgical procedures are therefore capable of limiting either subsequent immune rejection (by eliminating the endothelium layer) or subsequent thrombotic complications (by maintaining an endothelium layer). Clearly, a dichotomy exists as to which approach to pursue. Therefore, the clinical challenge for increasing patency rates in bypass grafting surgery is to develop procedures for the preparation of grafts that will limit both immune and thrombotic complications.

### SUMMARY OF THE INVENTION

The present invention is directed to an in vitro vascular graft composition comprising a tubular vascular graft surrounding an expanded stent wherein the expanded stent is opened over the endothelial layer of an appropriate length of tubular vascular tissue.

The present invention is also directed to vascular graft compositions described above wherein the stent is coated with a sustained release pharmaceutical compound such as, for example, an antibiotic such as Rapamycin (sirolimus), Actinomycin-D Taxol or QP2, a taxol derivitive.

Also described is a method of preparing a vascular graft material comprising the steps of: i) providing a tubular section of blood vessel; ii) destroying or inhibiting the ability of cells in the tubular section of blood vessel to cause an immune response upon retransplantation; iii) inserting a stent into the tubular section of blood vessel; iv) deploying the stent into an expanded form.

### DETAILED DESCRIPTION OF THE INVENTION

For purposes of the present invention a stent is a tubular support structure which fits within the lumen of a blood vessel and supports the walls of the blood vessel in a normal open configuration. Stents are well-known to the skilled artisan and are available in a myriad of different sizes, shapes, patterns and materials. The term stent encompasses wire mesh stents, compressed wire stents, roll type stents, coil type stents, as well as any other variety of expandable endovascular stent. The stent may be made of any bio-compatible material suitable for implantation into the body. The stents may be made, for example, of metal or polymer. The material may be designed for long or short duration in the patient's body after re-implantation and the material may be bioresorbable such as those materials described in U.S. Pat. No. 6,287,332 to Bolz et al., issued Sep. 11, 2001.

Moreover the stents can be of any length suitable to ensure the patency of the vascular tissue after re-implantation. For example, the stent may be shorter in length than the graft vascular tissue making re-implantation more facile by virtue of the vascular tissue extending beyond the end of the stent being easier to attach to the vascular tissue in the patient's body. In the alternative, the stent may be longer than the graft vascular tissue, providing a greater degree of support to ensure long term patency. Stents according to the present invention can range form 5 mm to about 50 mm in length, although most stents will fall into a length from about 10 mm to about 25 mm. An 18 mm stent can also be used in view of most arterial lesions being from about 8 mm to about 14 mm in length. Stents of an 18 mm length are currently marketed by a number of companies including Johnson and Johnson, Boston Scientific and Cook.

Stents according to the present invention will commonly have a diameter from about 1 mm to about 5 mm. Stents for use herein can also have a diameter of from about 2.5 mm to about 3.5 mm.

Stents according to the present invention may additionally act as controlled release dosage forms for administering a pharmaceutical having a beneficial impact on the patient. For purposes of the present invention, the term “beneficial agent” refers to a coating with one or more pharmaceutical such as an anti-proliferative agent, antimitotic agent, antibiotic, enzyme, proteins, hormones, steroids, anticoagulant, fibrinolytic, antiplatelet, antimigratory, antisecretory, anti-inflamatory, immunosuppressive, angiogenic agent, polymer and/or nitric oxide donor.

Preferred coatings include rapamycin, which may be used in combination with mycophenolic acid, Actinomycin D, heparin (Hepacoat), paclitaxel, batimastat, Resten-NG, Taxol, and QP2, a Taxol derivitive. These coatings may be used alone or in combination with another active agent.

A polymer encompasses any natural or synthetic homo or co-polymer which, either alone or in association with an active agent, shields vascular tissue from an adverse interaction with the stent. Such polymers include silicone carbide, polyethylene glycol and phosphorylcholine, for example. In addition the polymer may act as an excipient or vehicle with which to release active agent into the patients blood stream.

The amount of active agent may vary depending on the patients size and weight as will be appreciated by the skilled artisan. As a general proposition the amount of active agent can range from will be from. 1 mcg/mm² to about 250 mcg/mm² depending on the active agent to be employed, depending on the length and diameter of the stent, as well as the size and weight of patient. Dosages of from 0.5 to about 5 mcg/mm², and more preferably from about 1 to about 3.1 mcg/mm² can also be used.

The active agent can release the active agent over any period that will be sufficient to inhibit stenosis in the graft tissue typically a period from 1 hour to ninety days. Typically the coated stent will release from 1% to about 50% of the active agent in the first twenty four (24) hours and the remaining 99% to 50% in the following thirty (30) days to sixty (60) days. As the skilled artisan will appreciate, the coating can also release active agent from about 20% to about 40% in the first twenty four (24) hours and the remaining active agent over the next thirty (30) to sixty (60) days. In a preferred mode of the present invention, 25% to 35% of the one or more active agents will be released within the first twenty four hours and the remaining active ingredients over the following thirty (30) days.

The term vascular graft includes venous (vein) tissue, arterial (artery) tissue, or other man made or natural tissues which can be used in by-pass surgery or to create an anastomosis. The vascular graft may come from the patient to be operated on, i.e. the donor, an animal such as a pig, including genetically modified animals, such as genetically modified pigs, or a live human donor other than the patient, and human cadavers.

The term destroy means to compromise the vascular grafts ability to cause an immune response from the patient recipients immune system giving rise to rejection or restenosis of the graft.

Inhibit means reduce the ability of the vascular graft to cause an immune response from the patient recipients immune system giving rise to rejection of the graft.

The principle means by which the ability of the vascular graft material to cause an immune response is destroyed or inhibited is via the death or removal of the endothelial cells lining the vascular graft. The endothelial cells may be destroyed or removed via any means known in the art, including electrical, mechanical and chemical means. One method already being used during angioplasty with some success, is radiation. Any type of radiation sufficient to kill the endothelial cells and thus the ability of the vascular graft to cause an immune response may be used in the present invention.

The preferred method of destroying the endothelial layer is exposure of the endothelial cells to cold. Endothelial cells are sensitive to cold and immersion of the vascular tissue graft in a storage media and then storage in an environment below freezing (32° F. or 0° C.) destroys the endothelial layer of cells in the vascular tissue. Preferred temperature is at least −10° C. and most preferred is at −30° C.

The endothelial layer may also be destroyed by briefly passing a cold medium, such as water, saline solution or a frigid gas over the vascular graft and thereby the endothelial cells. Thus the ability of the native endothelial cells to promote a immune response can be destroyed, or substantially inhibited so that the graft material will not be rejected by the hosts body to the point of restenosis for a considerable period of time, including that period greater than three months.

The storage media used for preserving the vascular tissue can be any solution which will serve to maintain the vascular tissue for use without aggravating any factor which will result in stenosis once the vascular tissue in re-introduced into a patients body. A preferred storgae media is standard intravenous saline solution, although any saline solution suitable for the goals of the present invention may be used. Saline solutions with a saline concentration of from 0.05% to about 35% can be used. Saline solutions with a salt concentration of from about 1% to about 11% can also be used. A 9% saline solution is most preferred.

The storage media can also contain an anticoagulent. Anticoagulent agents are well known and any anticoagulent which will decresae the statistical probability of stenosis once the vascular tissue is re-introduced into a patient may be employed. Heparin is preferred and is introduced by dissolving an effective amount into the saline solution. An effective amount of anticoagulent will depend on the anticoagulent used and the size and weight of the vascular tissue. The skilled artisan will appreciate the known methods for introducing anticoagulent to the vascular tissue.

An antibiotic may also be employed in the storage media. Any antibiotic which furthers the goals of the present invention, i.e. lessening health risk to a patient receiving vascular tissue, can be used. Antibiotics such as penicillan, streptomycin, ampicillan, tetracyclin, amoxicillan and rapomycin may be used. Combinations of antibiotics have also been found to be effective for purposes of the present invention. The amount of antibiotic is well within the knowledge of the skilled practitioner depending on the kind of anti-biotic and the length and weight of tissue to be treated.

Vascular tissue according to the present invention can be stored in any manner conducive towards increasing the opportunity for successful implantation into the recipient patient. A preferred storage method is to add vascular graft and storage media to a plastic freezer bag and seal. Implantation and expansion of a stent can occur either prior to such storage, or after storage and before use of the vascular tissue in a patient.

The stent may be inserted into the vascular graft material in any way, and at any time before re-transplantation of vascular tissue into a patient, that is suitable to have the stent serve it's intended purpose of maintaining patency of graft material after insertion into a recipient. A stent deploying catheter serves this purpose well. In a preferred mode of the present invention the stent is inserted into the graft vascular tissue and deployed after the vascular tissue is thawed and within hours of being re-introduced into a patients body.

The following patents are herein incorporated by reference in their entirity as though set forth in full: U.S. Pat. No. 5,660,873; 5,782,910; 5,899,936; 6,074,659; 6,117,166; 6,261,318; 6,264,683; 6, 284,743; 6,287,332; 6,287,333; 6,293,959; and, 6,287,333.

### EXAMPLES

The following example is given to exemplify but not limit the claimed invention. All parts are by weight unless other specified.

### Example 1

Five pieces of immediately fresh cadavaric vascular tissue are harvested at the time of organ harvest by the transplant team. Each tissue sample is weighed, and measured for length and diameter, identified by blood grouping and patient name. Each tissue sample is irrigated with a 9% saline and solution containing 5,000 iu of heparin. Each tissue sample is then immersed in a separate flat bottomed freezer bag containing a mixture of between 10 and 35 cc of a 9% saline solution also containing 2,000,000 units of penicillan, 2 g of streptomycin and 5,000 iu heparin. Following immersion, the vascular tissue is stored in a freezer at −30° C. until six hours prior to implantation into a patient.

All patent, patent applications and other publications are herein incorporated by reference as though set forth in full.

The foregoing discussion describes merely exemplary embodiment illustrating the principles of the present invention, the scope of which is recited in the following claims. Those skilled in the yard will readily recognized from the description, claims, and drawings that numerous changes and modifications can be made without departing from the sprit and scope of the invention.

I/We claim:

1. An in vitro vascular graft composition comprising a tubular vascular graft surrounding an expanded stent.

2. A vascular graft composition according to claim 1 wherein the stent is coated with a sustained release pharmaceutical compound in an amount effective to prevent or inhibit substantial restenosis of the vascular graft material for a period of at least three months.

3. A vascular graft composition according to claim 2 wherein the stent is coated with an active agent selected from the group consisting of rapamycin, Actinomycin D, heparin, paclitaxel, batimastat, Resten-NG, Taxol, and QP2.

4. A vascular graft composition according to claim 1 to wherein the stent is coated with one or more pharmaceutically active agents selected from the group consisting of an anti-proliferative agent antimitotic agent, antibiotic, enzyme, proteins, hormones, steroids, anticoagulant agent, fibrinolytic agent, antiplatelet agent, antimigratory agent, antisecretory agent, anti-inflamatory agent, immunosuppressive agent, angiogenic agent, polymer and nitric oxide donor.

5. A vascular graft composition according to claim 1 wherein the stent is coated with an anti-thrombotic compound.

6. A vascular graft composition according to claim 1 wherein the stent is coated with two or more pharmaceutically active compounds.

7. A vascular graft composition according to claim 1 wherein the stent is coated with one or more pharmaceutically active agents and the amount of active agent is from about 0.1 mcg/mm² to about 250 mcg/mm² of the area of the stent.

8. A vascular graft composition according to claim 1 wherein the stent is coated with one or more pharmaceutically active agents and the amount of active agent is from about 0.5 to about 5 mcg/mm².

9. A method of preparing an in vitro vascular graft material comprising the steps of:
i) providing a tubular section of blood vessel;
ii) destroying or inhibiting the ability of cells in the tubular section of blood vessel to cause an immune response upon re-transplantation;
iii) deploying a stent into the tubular section of blood vessel.

10. A method of preparing a vascular graft material comprising the additional step;
iv) preparing the vascular graft composition for storage.
